(19) United States
(12) Patent Application Publication
(10) Pub. No.: US 2018/0333539 A1
(43) Pub. Date: Nov. 22, 2018

(54) SAFETY SYRINGE STRUCTURE

(71) Applicant: Sheng Yang International Technology Co., Ltd., New Taipei City (TW)

(72) Inventor: JEN-CHUAN KU, New Taipei City (TW)

(21) Appl. No.: 15/599,461

(22) Filed: May 18, 2017

(51) Int. Cl. A61M 5/32; A61M 5/31; A61M 5/34

(52) U.S. Cl. CPC A61M 5/322; A61M 5/3205; A61M 5/3221; A61M 5/3148; A61M 5/344; A61M 5/3129

(57) ABSTRACT

A safety syringe structure includes a combined structure of a syringe barrel, a needle retreater, a needle, and a plunger. The needle retreater and the plunger are slidably received in the syringe barrel. The needle retreater is arranged adjacent to the front end of the syringe barrel. The needle is coupled to the needle retreater. The plunger has an end extended to form a sharp tip and is provided with a plug facing the needle retreater. The sharp tip extends outside the plug in a direction toward the needle retreater. The sharp tip is movable with the plunger toward the front end of the syringe barrel to pierce into and couple with the through hole of the needle retreater; and the sharp tip is movable with the plunger toward the rear end of the syringe barrel to drive, through the needle retreater, the needle into the channel.

### (a) TECHNICAL FIELD OF THE INVENTION

The present invention relates generally to a safety syringe structure, which allows a needle at a front end of a syringe barrel to retract into the syringe barrel after an injection operation is completed with the syringe so as to prevent the needle from puncturing people and thus improve safety thereof, and more particularly relates to a plunger tip that drives the needle back into the syringe barrel and a needle retreater.

### (b) DESCRIPTION OF THE PRIOR ART

A conventional injection syringe, after use, is often attached with body fluid on a needle located at a front end thereof and such body fluid may carry bacteria or viruses that could causes diseases on human bodies. To protect medical attendants or other people from being punctured by the needle and getting infected, a common practice of the medical attendants is to insert the used needle back into a cap. However, during inserting the needle back into the cap, due to inadvertency or tiredness, the medical attendants may accidently puncture themselves at the hand holding the cap with the needle and thus causing undesired infection.

In view of the above problems, the present invention aims to provide a solution that overcomes theses problems.

### SUMMARY OF THE INVENTION

The primary objective of the present invention is to provide a safety syringe structure, which comprises:

a syringe barrel, which has a front end, a rear end, and a channel, the channel being provided, on an inner wall thereof at a location adjacent to the rear end, with a stop, the channel being provided with a first necked opening having a reduced diameter at the front end, the first necked opening and the inner wall of the channel defining therebetween a first circumferential surface facing toward the rear end;

a needle retreater, which has a first end, a second end, and a through hole in communication with the first end and the second end, the first end being provided with a circumferential groove in an outer circumferential wall of the through hole such that inside walls of the circumferential groove respectively define an internal circumferential surface and an external circumferential surface, the internal circumferential surface being adjacent to the through hole, the external circumferential surface being provided with a threaded section, the through hole being provided, at the second end, with a second necked opening, the second necked opening and an inner wall of the through hole defining therebetween a second circumferential surface facing toward the first end, the needle retreater being slidably received in the channel such that the first end is positioned against the first circumferential surface of the syringe barrel to allow the first circumferential surface to prevent the needle retreater from moving out of the channel through the first necked opening, the second end facing toward the rear end of the syringe barrel;

a clog ring, which is fit to the second end of the needle retreater and is slidable with the needle retreater in the channel so that the clog ring provides liquid tightness between the second end of the needle retreater and the inner wall of the channel;

a needle, which comprises a mounting collar and a hollow needle shaft connected to the mounting collar, the mounting collar being formed, in an interior thereof, with a fitting hole, which is fit to the internal circumferential surface, the fitting hole being in communication with the needle shaft, the mounting collar being provided, on an outer wall thereof, with a projecting rim that is screwable onto the threaded section; and a plunger, which is slidable received in the channel, the plunger having an end extended to form a sharp tip and provided with a plug, the plug facing toward the second end of the needle retreater, the sharp tip extending, in a direction toward the second necked opening of the needle retreater, through and thus exposed outside the plug.

With the above, the sharp tip is movable with the plunger toward the front end of the syringe barrel to allow the sharp tip to pierce into and couple with the second necked opening of the needle retreater; and the sharp tip is movable with the plunger toward the rear end of the syringe barrel to allow the sharp tip to drive, through the needle retreater, the needle to move into the channel, while the stop prevents the needle retreater from moving out of the channel through the rear end of the syringe barrel. As such, the used needle can be securely and simply stowed to thereby improve safety after injection conducted with the syringe barrel. The event of puncturing other people occurring in a conventional syringe needle can be alleviated and prevented.

In the above safety syringe structure, the second end of the needle retreater is provided, on an outer wall thereof, with a circumferential recess, the clog ring being fit to and received in the circumferential recess.

In the above safety syringe structure, the inner wall of the channel is provided with an interfering section at a location close to the front end of the syringe barrel to interfere with a movement of the clog ring, the interfering section being one of a raised bead, a raised rib, and a raised flange.

In the above safety syringe structure, the plunger is provided, on one end thereof, with a weakened diameter-reduced section that is extended to form the sharp tip, so that when the plunger drives the plug to move out of the channel through the rear end of the syringe barrel, the weakened diameter-reduced section is allowed to break to leave the needle retreater and the needle to be held in the channel.

In the above safety syringe structure, the plunger, the weakened diameter-reduced section, and the sharp tip are integrally formed together as a unit structure.

In the above safety syringe structure, the sharp tip is provided with a barb facing the plug for retaining engagement with and thus coupling with the second circumferential surface.

In the above safety syringe structure, the sharp tip is made in the form of an arrowhead.

In the above safety syringe structure, the second necked opening is provided, on an inner wall thereof, with a plurality of thin plates circumferentially arranged and projecting radially toward a center of the second necked opening so that the thin plates surround and collectively define a center hole in the center of the second necked opening, a slit being formed between every two adjacent ones of the thin plates.

In the above safety syringe structure, the second necked opening is converged from the second end toward the first end and the second end is connected through a circumferential slope surface to the second necked opening.

In the above safety syringe structure, the stop comprises one of a raised bead, a raised rib, and a raised flange and the plug is formed with a concave section engageable with the stop.

The foregoing objectives and summary provide only a brief introduction to the present invention. To fully appreciate these and other objects of the present invention as well as the invention itself, all of which will become apparent to those skilled in the art, the following detailed description of the invention and the claims should be read in conjunction with the accompanying drawings. Throughout the specification and drawings identical reference numerals refer to identical or similar parts.

Many other advantages and features of the present invention will become manifest to those versed in the art upon making reference to the detailed description and the accompanying sheets of drawings in which a preferred structural embodiment incorporating the principles of the present invention is shown by way of illustrative example.

### BRIEF DESCRIPTION OF THE DRAWINGS

FIG. 1 is a perspective view illustrating a preferred embodiment of the present invention.

FIG. 2 is an exploded view of FIG. 1.

FIG. 3 is a cross-sectional view of FIG. 1.

FIG. 4 is an enlarged view of a portion of FIG. 3.

FIG. 5 is a perspective view, in an enlarged form, of a needle retreater of FIG. 2.

FIG. 6 is a cross-sectional view illustrating a condition of use shown in FIG. 3.

FIG. 7 is a cross-sectional view illustrating a condition of use subsequent to FIG. 6.

FIG. 8 is a cross-sectional view illustrating a condition of use subsequent to FIG. 7.

FIG. 9 is a cross-sectional view illustrating a condition of use subsequent to FIG. 8.

### DETAILED DESCRIPTION OF THE PREFERRED EMBODIMENTS

The following descriptions are exemplary embodiments only, and are not intended to limit the scope, applicability or configuration of the invention in any way. Rather, the following description provides a convenient illustration for implementing exemplary embodiments of the invention. Various changes to the described embodiments may be made in the function and arrangement of the elements described without departing from the scope of the invention as set forth in the appended claims.

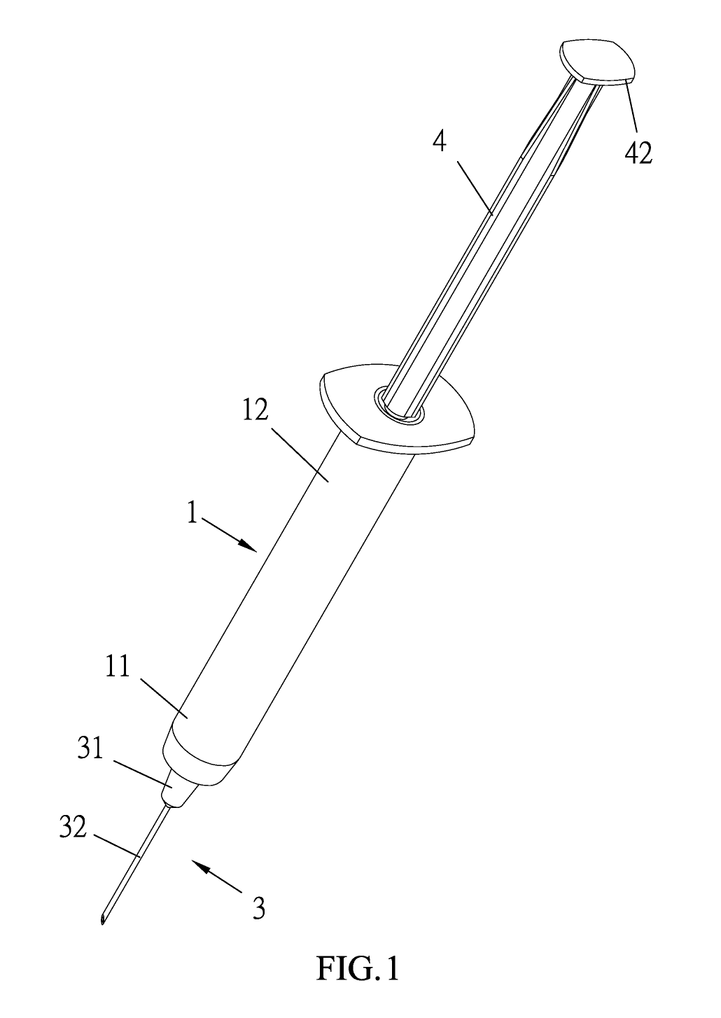

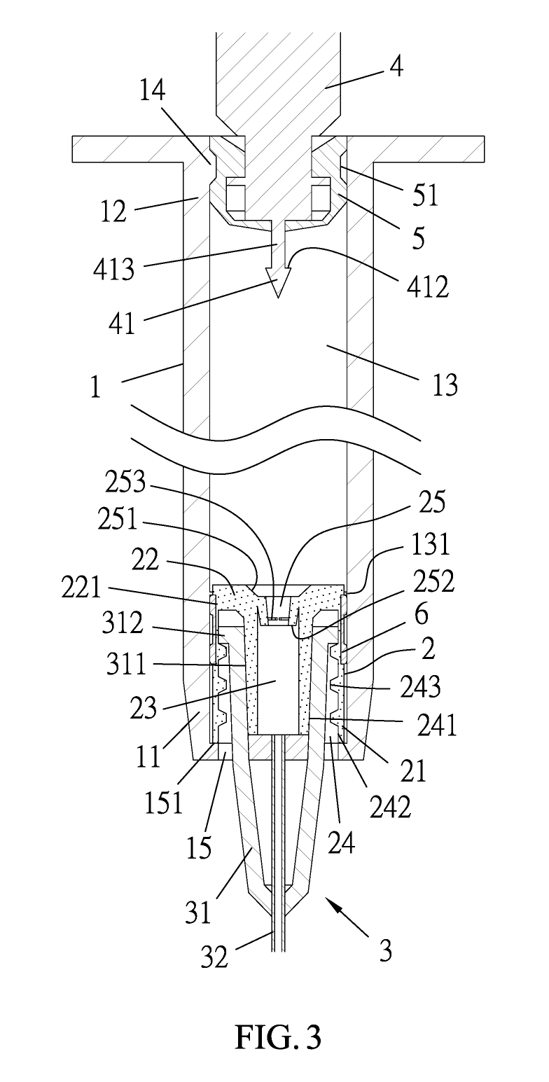

Referring to FIGS. 1-5, various views of an embodiment of the present invention are shown. These drawings show that a safety syringe structure according to the embodiment of the present invention comprises an assembled combination of a syringe barrel 1, a needle retreater 2, a needle 3, a plunger 4, and a clog ring 6. The syringe barrel 1 has two ends that respectively form a front end 11 and a rear end 12 and a channel 13 formed therein and in communication with the front end 11 and the rear end 12 of the syringe barrel 1. The channel 13 has an inside wall on which a stop 14 is formed at a location close to the rear end 12 of the syringe barrel 1. The stop 14 can be a raised bead, a raised rib, or a raised flange. The channel 13 is provided, at a portion thereof corresponding to the front end 11 of the syringe barrel 1, with a first necked opening 15 having a reduced diameter. A first circumferential surface 151 is defined between the first necked opening 15 and the inside wall of the channel 13 and faces toward the rear end 12 of the syringe barrel 1.

The needle retreater 2 has two ends that respectively form a first end 21 and a second end 22 and a through hole 23 formed therein and in communication with first end 21 and the second end 22 of the needle retreater 2. The first end 21 of the needle retreater 2 is formed with a circumferential groove 24 in an outer circumferential wall of the through hole 23 so that inside walls of the circumferential groove 24 respectively define an internal circumferential surface 241 and an external circumferential surface 242 facing and corresponding to each other. The internal circumferential surface 241 is adjacent to the through hole 23, and the external circumferential surface 242 is provided with a threaded section 243. The through hole 23 is provided, at the second end 22 of the needle retreater 2, with a second necked opening 25, and the second necked opening 25 is converging so as to have a diameter thereof reduced as extending in a direction from the second end 22 of the needle retreater 2 to the first end 21. The second end 22 of the needle retreater 2 is connected via a circumferential slope surface 251 to the second necked opening 25. A second circumferential surface 252 is defined between the second necked opening 25 and the through hole 23 and faces toward the first end 21 of the needle retreater 2. The needle retreater 2 is slidably mounted in the channel 13 of the syringe barrel 1 in such a way that the first end 21 of the needle retreater 2 is positioned against the first circumferential surface 151 of the syringe barrel 1 to allow the first circumferential surface 151 of the syringe barrel 1 to stop a movement of the needle retreater 2 through the first necked opening 15 to get out of the channel 13 and the second end 22 of the needle retreater 2 faces toward the rear end 12 of the syringe barrel 1, wherein an outer wall of the needle retreater 2 is not in tight engagement and contacting the inner wall of the channel 13 of the syringe barrel 1.

The second end 22 of the needle retreater 2 is provided, on the outer wall thereof, with a circumferential recess 221, and the clog ring 6 is received in and fit into the circumferential recess 221 so that the clog ring 6 slidably mounted, in combination with the needle retreater 2, in the channel 13 of the syringe barrel 1 with the clog ring 6 is tight engagement with and contacting the inner wall of the channel 13 of the syringe barrel 1, wherein the clog ring 6 provide liquid tightness between the outer wall of the second end 22 of the needle retreater 2 and the inner wall of the channel 13 of the syringe barrel 1 to prevent medicine from leaking through the first necked opening 15 of the channel 13 of the syringe barrel 1.

The needle 3 comprises a mounting collar 31 and a hollow needle shaft 32 connected to the mounting collar 31. The mounting collar 31 is formed, in an interior thereof, with a fitting hole 311 for fitting to the internal circumferential surface 241 of the needle retreater 2. The fitting hole 311 is in communication with the needle shaft 32. Further, the mounting collar 31 is provided, on an outer wall thereof, with a projecting rim 312, so that the mounting collar 31 can be screwed, through the projecting rim 312, to the threaded section 243 of the needle retreater 2.

The plunger 4 is slidably received in the channel 13. The plunger 4 has an end, which is formed with a sharp tip 41 extending therefrom and is provided with a plug 5 made of silicone rubber or rubber. The plug 5 faces toward the second end 22 of the needle retreater 2. The sharp tip 41 extends, perhaps through piercing, in a direction toward the second necked opening 25 (or the circumferential slope surface 251) of the needle retreater 2, through the plug 5 so that the sharp tip 41 is exposed and located outside the plug 5. The sharp tip 41 can be made in the form of an arrowhead. The sharp tip 41 comprises a barb 412 facing toward the plug 5 and engageable with the second circumferential surface 252 of the second necked opening 25. The plug 5 is provided, on an outer circumference thereof, with a concave section 51, in which the stop 14 of the syringe barrel 1 is receivable and engageable therewith to prevent the plug 5 from moving out of and separating from the channel 13. The plunger 4 has an opposite end on which a push section 42 is formed for operating by an operator.

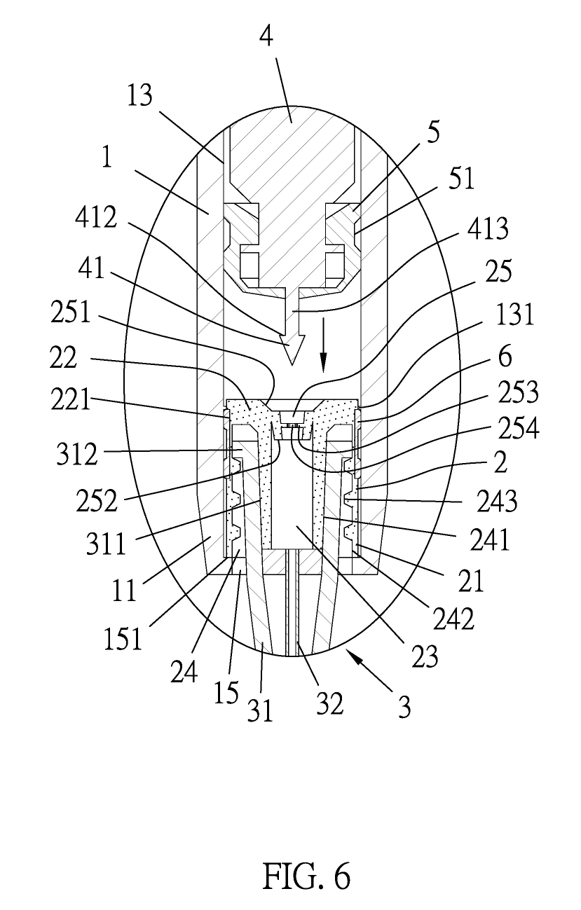

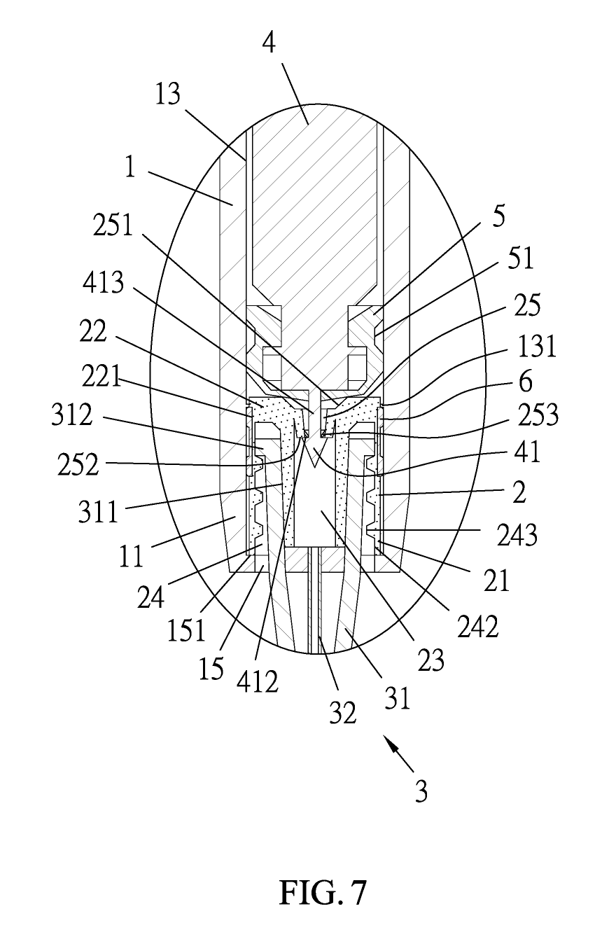

Referring to FIG. 6, when the operator operates the plunger 4 to drive the plug 5 to move along the channel 13 in a direction toward the front end 11 of the syringe barrel 1, the plug 5 forces medicine contained in the channel 13 to move through the second necked opening 25 and the through hole 23 of the needle retreater 2 and the needle shaft 32 of the needle 3 to get the outside or into a vessel of a human body. Referring to FIG. 7, with the medicine contained in the syringe barrel 1 being ejected, the sharp tip 41 moves with the plunger 4 toward the front end 11 of the syringe barrel 1, so that the sharp tip 41 passes over the circumferential slope surface 251 and inserts into the second necked opening 25 of the needle retreater 2, wherein the barb 412 of the sharp tip 41 moves through the second necked opening 25 and gets in retaining engagement with the second circumferential surface 252.

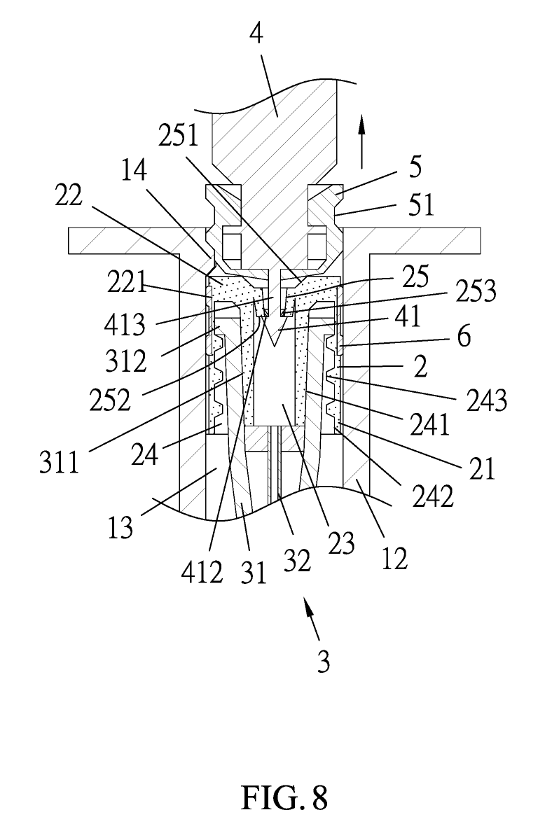

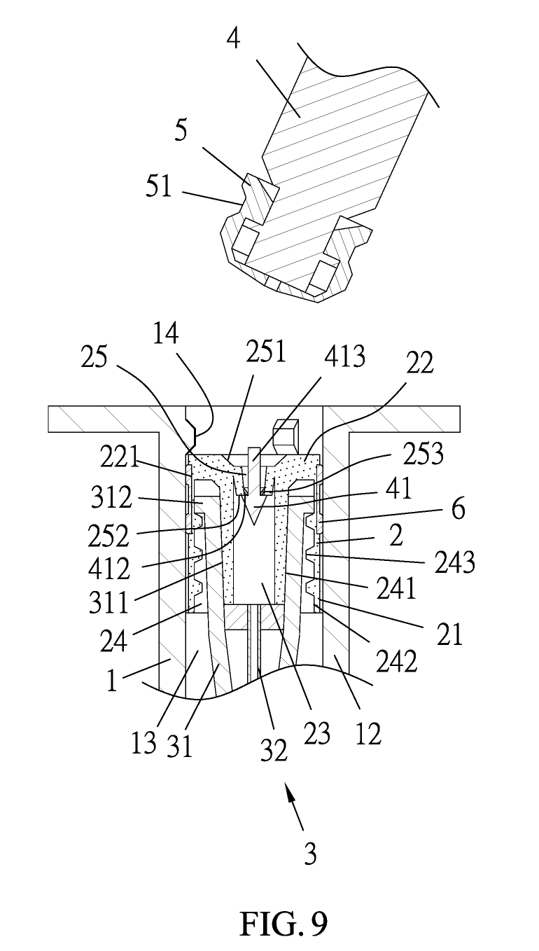

Referring to FIG. 8, when the operator operates the plunger 4 to drive the plug 5 to move along the channel 13 toward the rear end 12 of the syringe barrel 1, the sharp tip 41 is moved with the plunger 4 toward the rear end 12 of the syringe barrel 1, so that the sharp tip 41 forces, via the sharp tip 41 thereof, the second circumferential surface 252 of the needle retreater 2 to move in a direction toward the rear end 12 of the syringe barrel 1 and thus, the sharp tip 41 drives, via the needle retreater 2, the needle 3 (including the mounting collar 31 and the needle shaft 32) to move into the channel 13. Referring to FIG. 9, when the plug 5 reaches the rear end 12 of the syringe barrel 1, the stop 14 of the channel 13 provides interference with the second end 22 of the needle retreater 2 to prevent the needle retreater 2 from moving out of channel 13 through the rear end 12 of the syringe barrel 1.

As such, the used needle 3 can be securely received in the interior of the syringe barrel 1 to thereby improve safety after injection conducted with the syringe barrel 1. The event of puncturing other people occurring in a conventional syringe needle can be alleviated and prevented. Further, it only needs to operate the plunger 4 to move toward the rear end 12 of the syringe barrel 1 to carry out an operation of retreating the needle 3 back into the syringe barrel 1 so that the operation can be easily carried out.

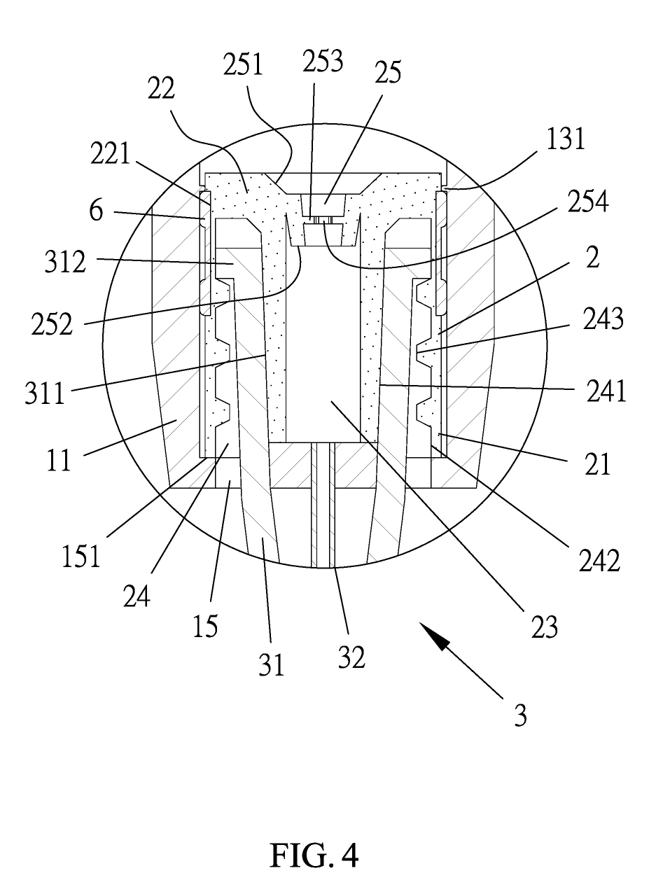

As shown in FIG. 4, in a feasible example, the channel 13 of the syringe barrel 1 is provided with an interfering section 131 on the inner wall thereof at a location close to the front end 11 of the syringe barrel 1. The interfering section 131 can be a raised bead, a raised rib, or a raised flange that interferes with a movement of the clog ring 6 in the channel 13 toward the front end 11 or the rear end 12 of the syringe barrel 1 so as to retain the needle retreater 2 in position. Particularly, During a process when an operator is operating the plunger 4 (or the plug 5) to make the needle 3 draw medicine from the outside into the channel 13 of the syringe barrel 1, at this moment, the sharp tip 41 is not inserted into and coupled with the second necked opening 25 of the needle retreater 2, and the interfering section 131 constrains the clog ring 6 and the needle retreater 2 from moving toward the rear end 12 of the syringe barrel 1; and when the sharp tip 41 has been inserted into and coupled with the second necked opening 25 of the needle retreater 2 to drive the needle retreater 2 and the clog ring 6 to move toward the rear end 12 of the syringe barrel 1, the sharp tip 41 forces the clog ring 6 to overcome a frictional forces induced by the interfering section 131 to allow the needle retreater 2 and the clog ring 6 to smoothly move toward the rear end 12 of the syringe barrel 1.

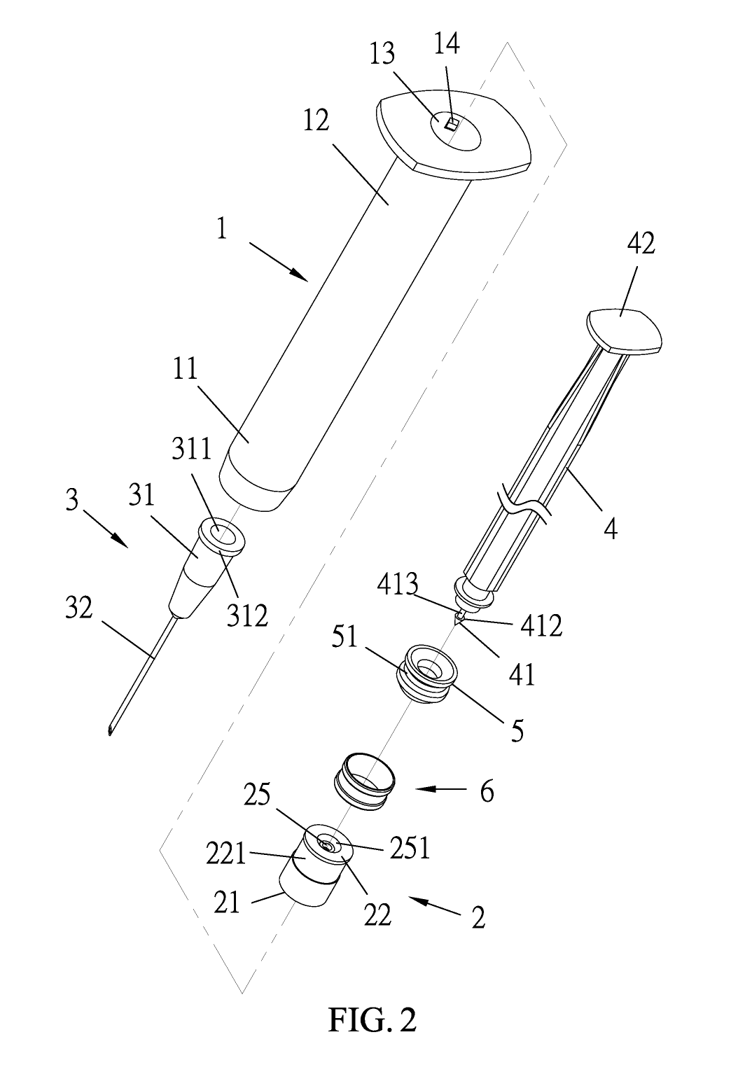

Referring to FIGS. 2, 8, and 9, in another feasible example, the plunger 4 is provided, on an end thereof, with a weakened diameter-reduced section 413 that is extended to form the sharp tip 41. The plunger 4, the weakened diameter-reduced section 413, and the sharp tip 41 can be made of a plastic material and integrally formed with each other to make the overall structure simple. As such, the sharp tip 41 (or the weakened diameter-reduced section 413) is allowed to directly pierce into the second necked opening 25 of the needle retreater 2 without extension through any object (such as the plug 5). Since the material (silicone rubber or rubber) that makes the plug 5, such as silicone rubber or rubber, may have a strength that is greater than the material (plastics) that makes the sharp tip 41 and the weakened diameter-reduced section 413, if the sharp tip 41 has to first pierce through the plug 5 and then pierce into the second necked opening 25 of the needle retreater 2, it might be possible that the sharp tip 41 may hardly pierce through the plug 5 or may cause the weakened diameter-reduced section 413 to break or crack due to being pressurized by the plug 5. It is appreciated that due to the weakened diameter-reduced section 413 having the characteristics of being easily broken or cracked, when the plunger 4 drives the plug 5 to move out of the channel 13 through the rear end 12 of the syringe barrel 1, the plunger 4 can be pulled sideways or transversely toward one side of the syringe barrel 1 to efficiently and easily break the weakened diameter-reduced section 413, thereby separating the sharp tip 41 and the plunger 4 from each other to leave the needle retreater 2 and the needle 3 held inside the channel 13. The operation is simple and the overall length of the syringe barrel 1 is reduced to facilitate collection and processing of medical waste.

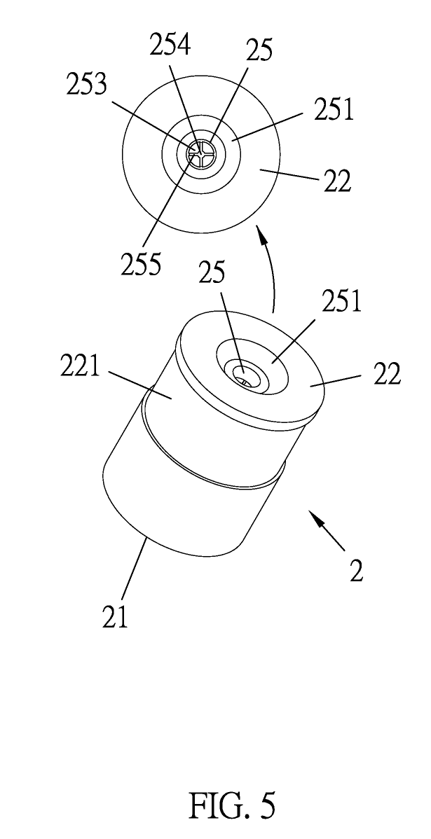

As shown in FIGS. 4 and 5, as a further feasible example, the second necked opening 25 is provided, on an inner wall thereof, with a plurality of thin plates 254 that are arranged circumferentially and projecting radially in a direction toward a center of the second necked opening 25 so that the thin plates 253 circumferentially surround the center of the second necked opening 25 to define a central hole 254, and a slit 255 is defined between every two adjacent ones of the thin plates 253. As shown in FIGS. 6-8, when the sharp tip 41 is inserted into the second necked opening 25 of the needle retreater 2, the sharp tip 41 moves through the central hole 254 to get into the through hole 23 with the sharp tip 41 pushes the thin plates 253 to deflect in a direction toward the through hole 23 so as to allow the thin plates 253 to tightly engage and enclose the sharp tip 41 and the weakened diameter-reduced section 413, thereby improving stability of the sharp tip 41 inserted into and coupled with the second necked opening 25 of the needle retreater 2 and preventing the sharp tip 41 and the second necked opening 25 from separating from each other.

It will be understood that each of the elements described above, or two or more together may also find a useful application in other types of methods differing from the type described above.

While certain novel features of this invention have been shown and described and are pointed out in the annexed claim, it is not intended to be limited to the details above, since it will be understood that various omissions, modifications, substitutions and changes in the forms and details of the device illustrated and in its operation can be made by those skilled in the art without departing in any way from the claims of the present invention.

1: A safety syringe structure, comprising:
a syringe barrel, which has a front end, a rear end, and a channel, the channel being provided with a stop, which is located on an inner wall of the channel at a location adjacent to the rear end, the channel being provided with a first necked opening having a reduced diameter at the front end, the first necked opening and the inner wall of the channel defining therebetween a first circumferential surface facing toward the rear end;
a needle retreater, which has a first end, a second end, and a through hole in communication with the first end and the second end, the first end being provided with a circumferential groove in an outer circumferential wall of the through hole such that inside walls of the circumferential groove respectively define an internal circumferential surface and an external circumferential surface that face and are spaced from each other, such that the internal circumferential surface is adjacent to the through hole, the external circumferential surface being provided with a threaded section, the through hole being provided with a second necked opening, which is arranged at the second end of the needle retreater, the second necked opening and an inner wall of the through hole defining therebetween a second circumferential surface facing toward the first end, the needle retreater being slidably received in the channel such that the first end of the needle retreater is positioned against the first circumferential surface of the syringe barrel to allow the first circumferential surface to prevent the needle retreater from moving out of the channel through the first necked opening, the second end facing toward the rear end of the syringe barrel;
a clog ring, which is fit to the second end of the needle retreater and is slidable with the needle retreater in the channel so that the clog ring provides liquid tightness between the second end of the needle retreater and the inner wall of the channel;
a needle, which comprises a mounting collar and a hollow needle shaft connected to the mounting collar, the mounting collar being formed with a fitting hole in an interior thereof, the fitting hole being fit to the internal circumferential surface of the circumferential groove of the needle retreater, the fitting hole being in communication with the needle shaft, the mounting collar being provided with a projecting rim, which is arranged on an outer wall of the mounting collar, with a such that the projecting rim is screwable onto the threaded section; and
a plunger, which is slidably received in the channel, the plunger having an end extended to form a sharp tip, the plunger being provided with a plug, which faces toward the second end of the needle retreater, the sharp tip extending in a direction toward the second necked opening of the needle retreater to penetrate through the plug and thus exposed outside the plug, wherein the sharp tip is movable with the plunger in a direction toward the front end of the syringe barrel to allow the sharp tip to pierce into and couple with the second necked opening of the needle retreater, and the sharp tip is also movable with the plunger in a direction toward the rear end of the syringe barrel to allow the sharp tip to drive, through the needle retreater, the needle to move into the channel, the stop preventing the needle retreater from moving out of the channel through the rear end of the syringe barrel;
wherein the inner wall of the channel is provided with an interfering section at a location close to the front end of the syringe barrel, the interfering section being set in engagement with an end of the clog ring so as to interfere with a movement of the clog ring and constrain the clog ring in position with respect to the inner wall of the channel by applying a frictional force to the clog ring, wherein when the plunger is moved in the direction toward the rear end of the syringe barrel, the plunger applies a driving force transmitted through the sharp tip to the needle retreater and the clog ring to overcome the frictional force that the interfering section applies to the clog ring to allow the needle retreater and the clog ring to pass over the interfering section and move toward the rear end of the syringe barrel for moving the needle into the channel.

2: The safety syringe structure as claimed in claim 1, wherein
the second end of the needle retreater is provided, on an outer wall thereof, with a circumferential recess, the clog ring being fit to and received in the circumferential recess.

3: The safety syringe structure as claimed in claim 1, wherein the interfering section comprises one of a raised bead, a raised rib, and a raised flange.

4: The safety syringe structure as claimed in claim 1, wherein the plunger is provided with a weakened diameter-reduced section on one end thereof, and the weakened diameter-reduced section is extended to form the sharp tip, so that when the plunger drives the plug to move out of the channel through the rear end of the syringe barrel, the weakened diameter-reduced section is allowed to break to leave the needle retreater and the needle to be held in the channel.

5: The safety syringe structure as claimed in claim 4, wherein the plunger, the weakened diameter-reduced section, and the sharp tip are integrally formed together as a unit structure.

6: The safety syringe structure as claimed in claim 1, wherein the sharp tip is provided with a barb facing the plug for retaining engagement with and thus coupling with the second circumferential surface.

7: The safety syringe structure as claimed in claim 1, wherein the sharp tip is made in the form of an arrowhead.

8: The safety syringe structure as claimed in claim 1, wherein the second necked opening is provided with a plurality of thin plates that are located on an inner wall of the second necked opening and are circumferentially arranged and projecting radially toward a center of the second necked opening so that the thin plates surround and collectively define a center hole in the center of the second necked opening, a slit being formed between every two adjacent ones of the thin plates.

9: The safety syringe structure as claimed in claim 1, wherein the second necked opening is converged from the second end toward the first end and the second end is connected through a circumferential slope surface to the second necked opening.

10: The safety syringe structure as claimed in claim 8, wherein the second necked opening is converged from the second end toward the first end and the second end is connected through a circumferential slope surface to the second necked opening.

11: The safety syringe structure as claimed in claim 1, wherein the stop comprises one of a raised bead, a raised rib, and a raised flange and the plug is formed with a concave section engageable with the stop.
